(19) United States
(12) Patent Application Publication
(10) Pub. No.: US 2024/0350710 A1
(43) Pub. Date: Oct. 24, 2024

(54) MULTIAXIAL ARTIFICIAL MUSCLE TISSUE, AND METHOD AND STRUCTURE FOR FORMING SAME

(71) Applicant: POSTECH RESEARCH AND BUSINESS DEVELOPMENT FOUNDATION, Pohang-si (KR)

(72) Inventors: Jin Ah JANG, Pohang-si (KR); Dong Gyu HWANG, Goyang-si (KR)

(21) Appl. No.: 18/034,443

(22) PCT Filed: Nov. 1, 2021
(86) PCT No.: PCT/KR2021/015609

§ 371 Date: Apr. 28, 2023

(30) Foreign Application Priority Data
Oct. 30, 2020 (KR) 10-2020-0143769
Nov. 1, 2021 (KR) 10-2021-0147809

(51) Int. Cl. A61L 27/38; A61F 2/08; A61L 27/52

(52) U.S. Cl. CPC A61L 27/3826; A61F 2/08; A61L 27/52; A61L 2430/20; A61L 2430/30

(57) ABSTRACT

A multiaxial artificial muscle tissue is provided. The multiaxial artificial muscle tissue according to an embodiment of the present invention is a multiaxial artificial muscle tissue contractible or stretchable about a plurality of axes and comprises: a first module formed by gelling a hydrogel including muscle cells and extending in a first axial direction so as to be contractible or stretchable in the first axial direction; and a second module formed by gelling the hydrogel and extending in a second axial direction to be contractible or stretchable in the second axial direction, wherein a portion of the hydrogel and a portion of the hydrogel which constitute the first module and the second module, respectively, are integrated with each other such that a portion of the first module and a portion of the second module are connected to each other.

### CROSS-REFERENCE TO RELATED APPLICATION

This application claims priority to and the benefit of Korean Patent Application No. 10-2020-0143769, filed on Oct. 30, 2020, and Korean Patent Application No. 10-2021-0147809, filed on Nov. 1, 2021, the disclosures of which are incorporated herein by reference in their entirety.

### TECHNICAL FIELD

The present invention relates to a multiaxial artificial muscle tissue, a method and a structure for forming the same, and more specifically to a multiaxial artificial muscle tissue that is contractible or stretchable around a plurality of axes, and a method and a structure for forming the same.

### BACKGROUND ART

Muscle tissue is a tissue that contracts in response to nerve stimulation, and it is structurally divided into striated muscle and smooth muscle, and more specifically classified into skeletal muscle, cardiac muscle and smooth muscle. In particular, skeletal muscle and cardiac muscle tissues form a long columnar shape and contract in the longitudinal direction of a cell.

This muscle tissue has certain directionality in order to efficiently generate a contractile force. For example, skeletal muscle tissue is aligned in one direction, and it is attached to the bone to contract and move the body. In addition, the cardiac muscle tissue plays a role in circulating blood throughout the body through a contractile movement, and unlike the skeletal muscle tissue, it has a non-linear chamber shape as illustrated in FIG. 1. In order to efficiently produce contractile force, myocardial cells are aligned in one direction to form tissue, but they are aligned in different directions from the pericardium to the endocardium of the left ventricle and form a spiral structure. This allows the ventricle to contract sufficiently and allows blood to circulate better throughout the body.

Meanwhile, artificial muscle tissue is being studied to replace damaged skeletal muscle and cardiac muscle or to help regeneration. In addition, it is being used as a platform for helping the understanding of diseases or researching new treatment methods by simulating the pathophysiological characteristics of each muscle tissue in the body in vitro.

As described above, since muscle is a tissue with directionality, in terms of fabricating an artificial muscle tissue, imparting the directionality of cells to simulate the environment of actual muscle tissue plays an important role in improving tissue function.

Currently, the most widely used method to impart directionality to a three-dimensional artificial muscle tissue is to place a hydrogel including muscle cells in a mold and produce the tissue. The hydrogel placed in the mold has a property of contracting as the tissue is formed. In this case, if a tensile force is applied in the opposite direction to the contracting tissue by using a post or an elastic wire inside the mold, the cells in the tissue have directionality according to the tensile force.

However, in the existing method, the size and structure of the mold are limited, and thus, the size and directionality of the tissue that can be fabricated are limited. In particular, in the case of the existing method, as illustrated in FIG. 1, it is difficult to simulate the heart muscle having various directions, and thus, there is a limitation in simulating the shape of the actual cardiac muscle tissue.

### DISCLOSURE

### Technical Problem

The present invention has been devised in view of the above points, and an object of the present invention is to provide a multiaxial artificial muscle tissue having various directions.

In addition, another object of the present invention is to provide a method and a structure for forming a multiaxial artificial muscle tissue such that the multiaxial artificial muscle tissue can be easily implemented.

### Technical Solution

In order to achieve the above-described objects, the present invention provides a multiaxial artificial muscle tissue which is a multiaxial artificial muscle tissue that is contractible or stretchable about a plurality of axes, including a first module which is formed by gelling a hydrogel including muscle cells and extends in a first axis direction so as to be contractible or stretchable in the first axis direction; and a second module which is formed by gelling the hydrogel and extends in a second axis direction to be contractible or stretchable in the second axis direction, wherein a portion of the hydrogel and a portion of the hydrogel which constitute the first module and the second module, respectively, are integrated with each other such that a portion of the first module and a portion of the second module are connected to each other.

In this case, the muscle cells may include cardiomyocytes.

In this case, the muscle cells may include skeletal muscle cells.

In this case, the first axis and the second axis may be disposed to cross each other.

In this case, the first axis and the second axis may be disposed to be parallel to each other.

In this case, the multiaxial artificial muscle tissue may further include a third module which is formed by gelling the hydrogel and extends in a third axis direction so as to be contractible or stretchable in the third axis direction, wherein a portion of the hydrogel and a portion of the hydrogel which constitute the first module and the third module, respectively, are integrated with each other such that a portion of the first module and a portion of the third module are connected to each other.

In this case, the first module and the second module may be integrally formed by using a single frame in a multi-axis structure including the first axis and the second axis.

In this case, after the first module and the second module are primarily cross-linked at positions that are spaced apart from each other, the first module may be moved to the second module and connected to each other.

In this case, a portion of the first module may be disposed above the second module.

In this case, the first module and the second module may be disposed to contact each other on the same plane.

Meanwhile, the present invention provides a method for forming a multiaxial artificial muscle tissue which is a method for forming the above-described multiaxial artificial muscle tissue, including the steps of forming the first module by using a first frame; forming the second module by using a second frame which is positioned to be spaced apart from the first frame; inducing the muscle cells included in the first module and the second module to be aligned along the first axis direction and the second axis direction, respectively; moving the first module or the second module such that the first module and the second module come into contact with each other; and inducing a portion of the hydrogel and a portion of the hydrogen constituting the first module and the second module, respectively, to be integrated with each other.

In this case, the step of moving the first module to come into contact with the second module may include the steps of inverting the first frame; and stacking the first frame on the second frame such that the first module and the second module are in contact with each other.

In this case, the step of moving the first module to come into contact with the second module may include the steps of separating the first module from the first frame and fixing to a third frame; and separating the second module from the second frame and fixing to the third frame.

Meanwhile, the present invention provides a structure which is a structure for forming the above-described multiaxial artificial muscle tissue, including a side wall portion which has a side wall to partition an accommodation space in which a predetermined amount of the hydrogel is accommodated; and a column portion which includes first to fourth columns that are disposed to be spaced apart from each other in the accommodation space and supported by the side wall portion, wherein the side wall portion includes a plurality of bent portions formed by being drawn into the accommodation space, and wherein the first to fourth columns are disposed such that a first imaginary line connecting the first column and the second column and a second imaginary line connecting the third column and the fourth column cross each other.

In this case, the first column and the second column may be disposed to be spaced apart from each other at an interval that facilitates fixation in a state where both end portions of the first module in a primarily cross-linked state are inserted, and the third column and the fourth column may be disposed to be spaced apart from each other at an interval that facilitates fixation in a state where both end portions of the second module in a primarily cross-linked state are inserted.

In addition, the present invention provides a structure which is a structure for forming the above-described multiaxial artificial muscle tissue, including a side wall to partition an accommodation space from the external space, wherein the side wall includes a plurality of bent portions that are drawn into the accommodation space such that a plurality of basic frames having a rectangular shape can be stacked and disposed in a state of crossing each other in the accommodation space, and has a thickness that is greater than or equal to the stack height of the plurality of basic frames.

### Advantageous Effects

The multiaxial artificial muscle tissue according to an exemplary embodiment of the present invention can implement a multiaxial artificial muscle tissue that is contractible or stretchable around a plurality of axes by integrating and connecting to each other portions of the basic modules that are arranged to have directionality with respect to a specific axis, respectively.

In addition, the multiaxial artificial muscle tissue according to an exemplary embodiment of the present invention can increase the degree of freedom in fabricating an artificial muscle tissue by freely connecting basic modules in various structures.

In addition, the method for forming a multiaxial artificial muscle tissue according to an exemplary embodiment of the present invention can form the multiaxial artificial muscle tissue in various ways by considering the structure or manufacturing environment, such as a method of moving the basic frame itself or a method of separating modules from the basic frame and connecting the same.

In addition, the structure for forming a multiaxial artificial muscle tissue according to an exemplary embodiment of the present invention can perform various functions in a complex manner according to the method for forming a multiaxial artificial muscle tissue.

### DESCRIPTION OF DRAWINGS

FIG. 1 is an explanatory diagram for explaining the complexity of the heart muscle.

FIG. 2 is a view illustrating the multiaxial artificial muscle tissue according to an exemplary embodiment of the present invention.

FIG. 3 is a view illustrating a state in which the first module of FIG. 2 is actually formed by using a basic frame.

FIG. 4 is a view illustrating the basic frame of FIG. 3.

FIG. 5 is a view illustrating a state in which the first module and the second module of FIG. 2 are actually connected.

FIG. 6 is a view illustrating a state in which the first to third modules are connected by using the structure for forming a multiaxial artificial muscle tissue according to an exemplary embodiment of the present invention.

FIGS. 7 and 8 are views illustrating various examples of the structure for forming a multiaxial artificial muscle tissue according to an exemplary embodiment of the present invention.

FIGS. 9 to 11 are flowcharts illustrating each step of the method for forming a multiaxial artificial muscle tissue according to an exemplary embodiment of the present invention.

FIG. 12 is a view for explaining the stacking of a basic frame on the structure for forming a multiaxial artificial muscle tissue according to an exemplary embodiment of the present invention.

FIG. 13 is an explanatory diagram sequentially illustrating a process in which cells included in a module constituting the multiaxial artificial muscle tissue are aligned according to an exemplary embodiment of the present invention.

FIG. 14 is an explanatory diagram sequentially illustrating a process in which the multiaxial artificial muscle tissue according to an exemplary embodiment of the present invention is integrated.

FIG. 15 is an image obtained by photographing a multiaxial artificial muscle tissue over time after fluorescence staining in order to observe the cell alignment of cells included in the multiaxial artificial muscle tissue according to an exemplary embodiment of the present invention.

### MODES OF THE INVENTION

Hereinafter, with reference to the accompanying drawings, the exemplary embodiments of the present invention will be described in detail so that those of ordinary skill in the art can easily practice the present invention. The present invention may be embodied in many different forms and is not limited to the exemplary embodiments described herein. In order to clearly describe the present invention in the drawings, parts that are irrelevant to the description are omitted, and the same reference numerals are assigned to the same or similar elements throughout the specification. In addition, the size or shape of the components shown in the drawings may be exaggerated for clarity and the convenience of explanation.

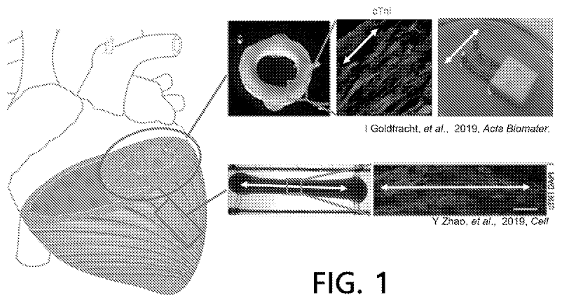

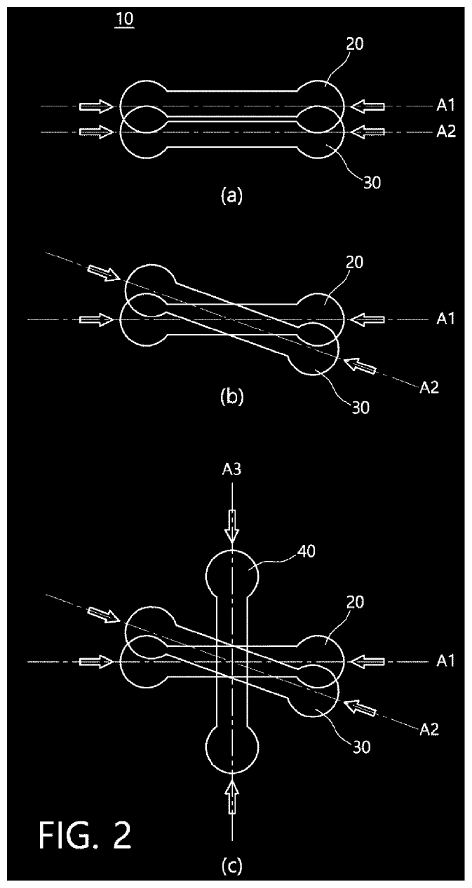

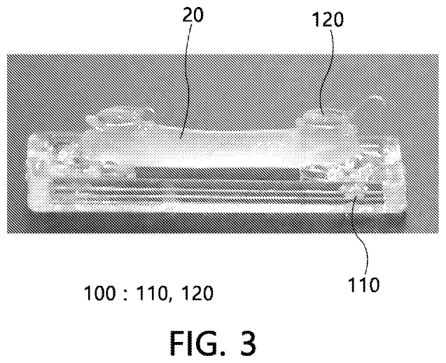

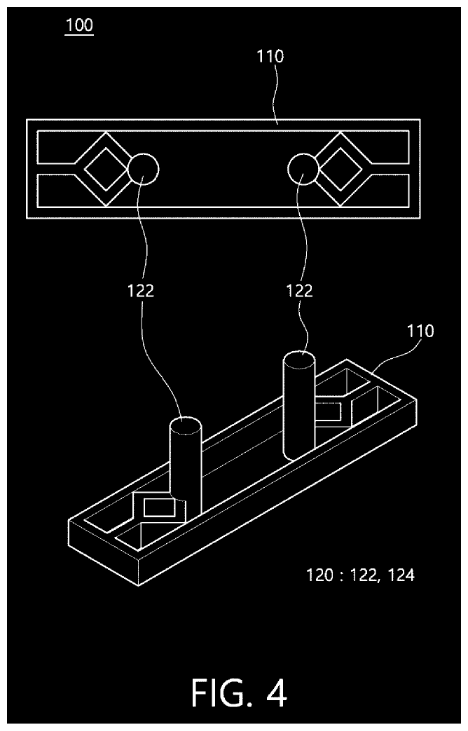

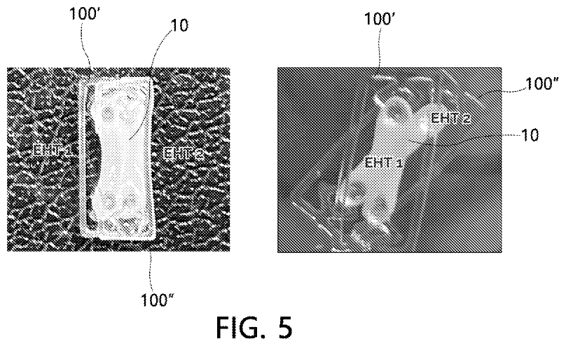

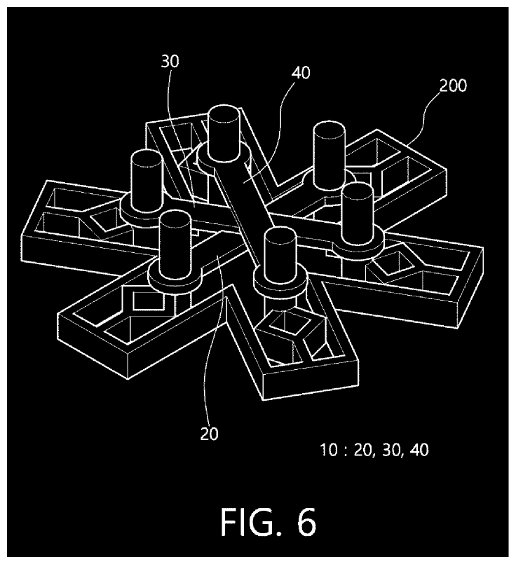

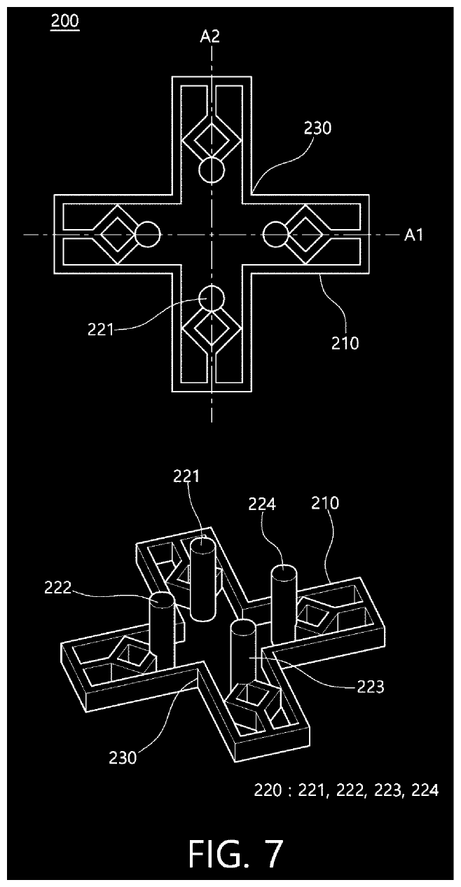

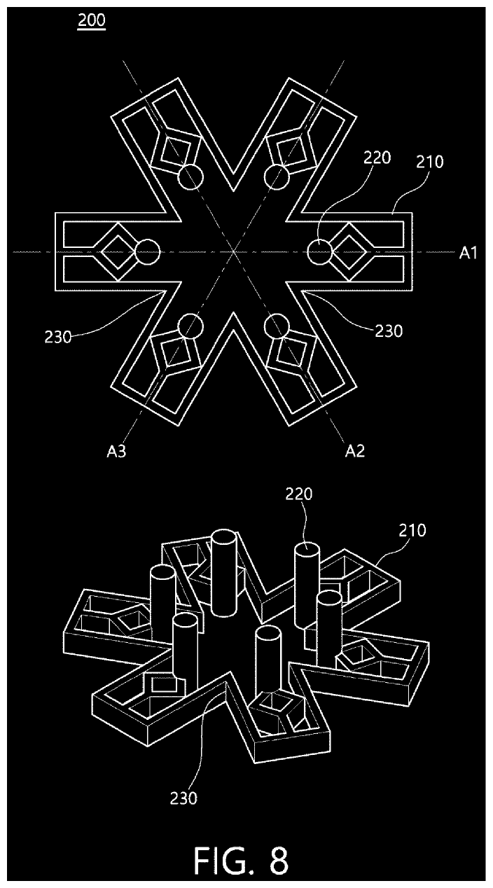

FIG. 1 is an explanatory diagram for explaining the complexity of the heart muscle. In addition, FIG. 2 is a view illustrating the multiaxial artificial muscle tissue according to an exemplary embodiment of the present invention, and FIG. 3 is a view illustrating a state in which the first module of FIG. 2 is actually formed by using a basic frame. In addition, FIG. 4 is a view illustrating the basic frame of FIG. 3, FIG. 5 is a view illustrating a state in which the first module and the second module of FIG. 2 are actually connected, and FIG. 6 is a view illustrating a state in which the first to third modules are connected by using the structure for forming a multiaxial artificial muscle tissue according to an exemplary embodiment of the present invention. Next, FIGS. 7 and 8 are views illustrating various examples of the structure for forming a multiaxial artificial muscle tissue according to an exemplary embodiment of the present invention.

The multiaxial artificial muscle tissue 10 according to an exemplary embodiment of the present invention is an artificial muscle tissue formed by simulating a living muscle to replace a damaged biological muscle or to assist in the regeneration of a damaged muscle. In this case, the multiaxial artificial muscle tissue according to an exemplary embodiment of the present invention may contract or stretch around a plurality of axes of two or more axes, unlike the related art in which contraction or tension is possible only in a limited direction toward a certain uniaxial axis. Through this, the multiaxial artificial muscle tissue according to an exemplary embodiment of the present invention may simulate the motility of a complex muscle tissue having directionality in various directions, such as the cardiac muscle or skeletal muscle of FIG. 1. Hereinafter, the main constitution and effects of the multiaxial artificial muscle tissue according to an exemplary embodiment of the present invention will be first described in detail, and then, the method and structure for forming a multiaxial artificial muscle tissue will also be described.

Specifically, referring to FIG. 2, the multiaxial artificial muscle tissue 10 according to an exemplary embodiment of the present invention may include a first module 20 and a second module 30 in the form of being connected to each other.

First of all, the first module 20 is a portion constituting a part of the multiaxial artificial muscle tissue, and it may be formed by gelling a hydrogel including muscle cells.

In this case, as illustrated in FIG. 2, the first module 20 may be formed to extend in a first axis A1 direction so as to be contractible or stretchable in the first axis A1 direction. Specifically, the first module 20 may have directionality in the first axis direction by aligning the muscle cells included in the first module along the first axis A1 direction. Through this, the first module 20 may enable contraction or tension in the first axis A1 direction in a multiaxial artificial muscle tissue having directionality in a plurality of directions.

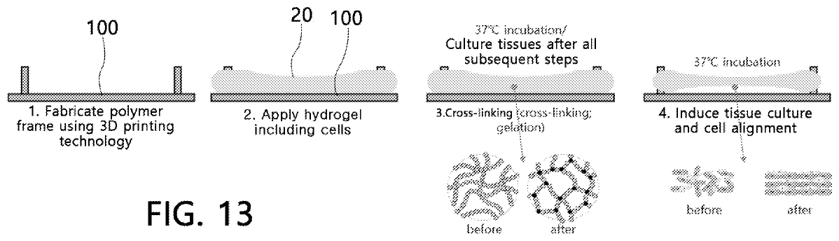

In an exemplary embodiment of the present invention, the first module 20 may be formed by gelation of a hydrogel including muscle cells. In other words, as illustrated in FIG. 3, the first module 20 may be formed such that the hydrogel in a fluidized state without a certain shape initially forms cross-linking over time and contracts as the tissue matures to have a certain shape (refer to FIG. 13).

For example, the hydrogel may be formed by decellularizing a pig's heart tissue, and the concentration thereof may be 0.6% (w/v). However, this is only an illustrative example, and the hydrogel may be formed based on various muscle cells and concentrations.

Meanwhile, the first module 20 may be formed by using a basic frame 100 having a rectangular shape as illustrated in FIG. 4. In this case, the basic frame 100 is provided with a side wall portion 110 in a rectangular structure so as to partition a space in which the hydrogel can be accommodated therein as illustrated in the drawings, and a pair of columns 122, 124 facing each other may be provided on the inside. Meanwhile, standards of the basic frame may be variously changed according to specifications of the first module required in design, for example, an extension length and the like.

In this regard, when the hydrogel in a fluidized state is applied to the basic frame 100, the hydrogel may form the above-described cross-linking in a state where movement in the outward direction is restricted by the side wall portion 110. Afterwards, tissue is formed and may be contracted. In this case, a pair of columns 120 arranged side by side on the first axis may apply a tensile force to the muscle cells in the hydrogel, and accordingly, the muscle cells are aligned along the first axis A1 such that the first module 20 may have directionality (biasedness) with respect to a specific direction (first axis direction).

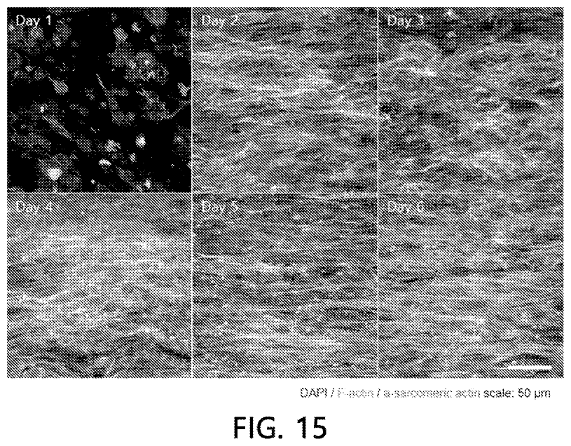

More specifically, the first module 20 may be aligned such that the cells have certain directionality according to the process illustrated in FIG. 13. That is, after the hydrogel including cells is applied in the basic frame, tissue culture may proceed at about 37° C. First of all, cells inside the hydrogel form cross-linking with adjacent cells as illustrated in the drawings. In this case, the time for performing cross-linking may be 30 minutes to 1 hour. Thereafter, cells interact with cells and the surrounding environment (hydrogel) inside the cross-linked muscle tissue, and tissue is formed through actions such as migration, proliferation and elongation. In addition, as time elapses, the cells may be aligned to have directionality in the first axis A1 direction under the influence of the tensile force by the columns 122, 124 of the frame. Through this, the first module 20 may have a directionality for a specific direction, and this directionality may become clearer as time elapses as illustrated in FIG. 15.

In this case, the hydrogel applied in the basic frame may include, for example, a total of 50×10⁶ cardiomyocytes and fibroblasts in a mixed state. Illustratively, the corresponding cardiomyocytes may be used by differentiating iPSCs (GM25256, Coriell Institute) into myocardium, and fibroblasts may be used with Human Cardiac fibroblasts (C-12375, Promocell). In addition, endothelial cells may be included together, and for endothelial cells, human umbilical vein endothelial cells; HUVEC (C-12200, Promocell) may be used. In addition, the ratio of cardiomyocytes to fibroblasts may be, for example, 9:1, and when endothelial cells are included, the ratio of cardiomyocytes to fibroblasts and endothelial cells may be 9:1:2. After the hydrogel is applied in the basic frame, the gelation of the hydrogel may be promoted by incubating in a 37° C. incubator for 40 minutes or more. However, it should be noted that the composition of the hydrogel constituting the first module and the method of forming the first module are not limited to the above-described examples.

The multiaxial artificial muscle tissue according to an exemplary embodiment of the present invention may include a second module 30 as another part constituting the multiaxial artificial muscle tissue in addition to the first module 20.

In this case, the second module 30 is formed by gelling a hydrogel in the same manner as the first module, and unlike the first module 20, it may be formed to extend in the second axis A2 direction. That is, the muscle cells included in the second module 30 are aligned along the second axis A2 direction, similarly to the alignment of the muscle cells of the first module 20, such that the second module 30 may have directionality in the second axis A2 direction. That is, the second module may be easily contracted or stretched in the second axis direction.

In addition to the above, the second module 30 is only different in that it extends along the second axis A2 direction, and most of the material, structure or method of forming may be the same as those of the first module 20. Accordingly, the additional description of the second module will be replaced with the description of the first module.

Meanwhile, in addition to the first module 20 and the second module 30, the multiaxial artificial muscle tissue 10 according to an exemplary embodiment of the present invention may further include a third module 40, a fourth module, . . . , and an N^{th} module. Through this, the multiaxial artificial muscle tissue according to an exemplary embodiment of the present invention may have directionality with respect to various axes, which will be described below.

Referring to FIGS. 2 and 5, the first module 20 and the second module 30 described above may be partially connected to each other. In this case, the first module and the second module may be connected to each other by integrating (integrally assembled) a portion of the hydrogel constituting each. Herein, the meaning of being connected may mean a state in which portions of the first module and the second module are integrated (integrally assembled) by forming a cell bond such that they can function organically as a single artificial muscle tissue.

As a specific example related to the connection, while the first module and the second module are each independently cross-linked to have directionality with respect to the first axis A1 and the second axis A2 through the above-described basic frames 100′, 100″, respectively, the first module and the second module may be connected to each other through a process in which any one is disposed to be in contact with the other one as shown in FIG. 2 such that the portions that are in contact with each other are integrated.

As described above, the first module 20 and the second module 30 are spaced apart from each other before being integrated with each other, and the alignment of muscle cells may be induced through tissue culture after cross-linking (this requires a minimum time of approximately 48 hours). In this case, the first module 20 and the second module 30 may independently align muscle cells toward the first axis and the second axis in a state that does not affect each other. Thereafter, after the cell alignment is formed, a process in which the first module and the second module are integrated (integrally assembled) in a state where they are in contact with each other may be performed.

As a result, the multiaxial artificial muscle tissue according to an exemplary embodiment of the present invention may function as a single muscle cell tissue by connecting the initially independently formed first module and second module to each other. Further, in the multiaxial artificial muscle tissue according to an exemplary embodiment of the present invention, as the first module 20 and the second module 30 each having directionality are connected, the first module 20 and the second module 30 may have directionality with respect to a plurality of axes including the first axis A1 and the second axis A2, and through this, it is possible to effectively simulate muscles having complex directionality such as the above-described cardiac muscle or skeletal muscle.

Meanwhile, as described above, when the multiaxial artificial muscle tissue is formed by forming the first module and the second module independently of each other and then connecting the same, it is possible to manufacture the multiaxial artificial muscle tissue having various structures according to the design.

More specifically, referring to FIG. 2, in the multiaxial artificial muscle tissue according to an exemplary embodiment of the present invention, various structures of artificial muscles may be freely implemented, such as connecting the first axis and the second axis to be parallel to each other as shown in FIG. 2(a), connecting to cross each other as shown in FIG. 2(b), or including a third module 40 as shown in FIG. 2(c), and through this, it is possible to secure a degree of freedom in manufacturing artificial muscle tissue. In particular, the multiaxial artificial muscle tissue according to an exemplary embodiment of the present invention may freely adjust the angle at which two modules intersect, thereby maximizing the degree of freedom. This will be described in more detail through a section for describing the method for forming a multiaxial artificial muscle.

In addition, the method of connecting the first module 20 and the second module 30 to each other may also be implemented in various ways. For example, as illustrated in FIG. 6, a portion of the first module 20 may be connected in a stacked form on a portion of the second module 30, and in contrast thereto, the first module 20 and the second module 30 may be connected in a state where they are disposed adjacent to each other on the same plane such that there is no step difference between the first module 20 and the second module 30.

In addition, although not illustrated in the drawings, in the multiaxial artificial muscle tissue according to an exemplary embodiment of the present invention, only modules in charge of a specific axis may be more emphasized by differentiating the standards of basic frames for forming the first module 20 and the second module 30.

In an exemplary embodiment of the present invention, the first module and the second module may be formed separately, respectively, to be described below by using the basic structure as described above, but, unlike this, as illustrated in FIGS. 7 and 8, the first module 20 and the second module 30 may be integrally formed by applying a hydrogel to a structure for forming the multiaxial artificial muscle tissue having a multiaxial structure. In this case, the hydrogel constituting the first module and the second module may be contracted by forming cross-linking at the same time, and a tensile force may be applied around the first axis A1 and the second axis A2. As a result, the first module and the second module may be formed in a structure which partially extends in the first axis and the second axis directions while sharing a partially overlapping area (central part in the drawings).

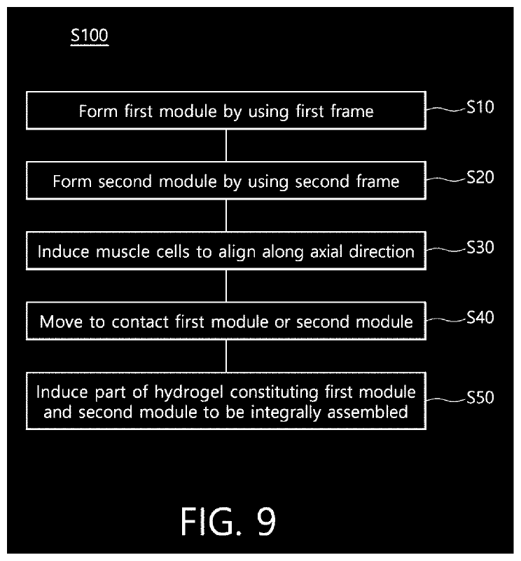

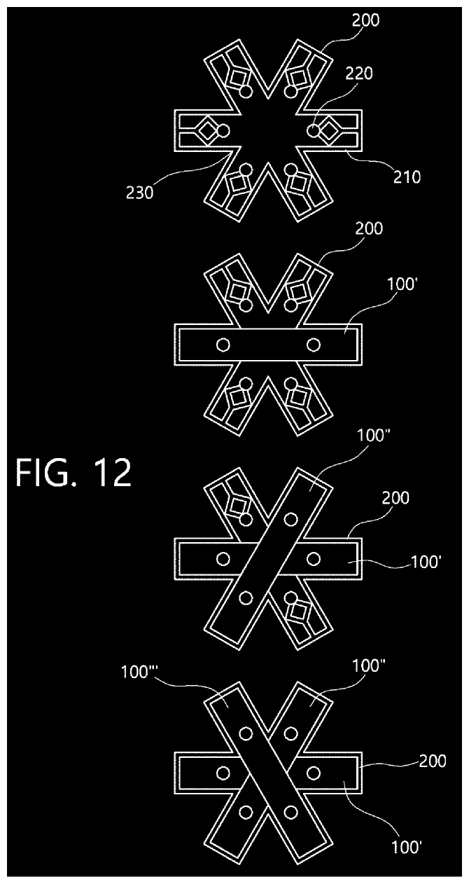

FIGS. 9 to 11 are flowcharts illustrating each step of the method for forming a multiaxial artificial muscle tissue according to an exemplary embodiment of the present invention, and FIG. 12 is a view for explaining the stacking of a basic frame on the structure for forming a multiaxial artificial muscle tissue according to an exemplary embodiment of the present invention.

Hereinafter, the method for forming a multiaxial artificial muscle tissue according to an exemplary embodiment of the present invention will be described with reference to the drawings.

As described above, the multiaxial artificial muscle tissue according to an exemplary embodiment of the present invention may be formed by a method of forming the first module and the second module independently and then connecting the same to each other (hereinafter, referred to as a ‘first method’) or a method of forming by applying a hydrogel to a structure having a multiaxial structure at once (hereinafter, referred to as a ‘second method’). In the following description, the first method S100 will be mainly described.

Referring to FIG. 9, in order to form the multiaxial artificial muscle tissue according to an exemplary embodiment of the present invention, the first module 20 may be formed by first applying a hydrogel to a first frame 100′ (same as the basic frame) S10. In addition, the second module 30 may be formed by applying a hydrogel to a second frame 100″, which is distinguished from the first frame S20.

Thereafter, the muscle cells included in the first module and the second module may be induced to be aligned along the first and second axis directions, respectively S30. That is, a predetermined amount of time may be waited such that the muscle cells in the hydrogel applied to the basic frame can mature. Through this process, the muscle cells in the hydrogel may be aligned to have directionality in one direction by applying a force in the uniaxial direction by a column portion, and cross-linking may be performed at the same time. The time required for such alignment may require a minimum time of within about 48 hours after the application of the hydrogel, but this is only an exemplary example, and it may be variously applied according to the characteristics of the hydrogel.

Next, the first module or the second module may be moved such that the first module and the second module are in contact with each other S40. This moving process may mean that the first module is disposed on a portion of the second module (stacking form) as described above, or it may mean that they are disposed to be adjacent to each other on the same plane.

Meanwhile, the moving process may be performed in various ways.

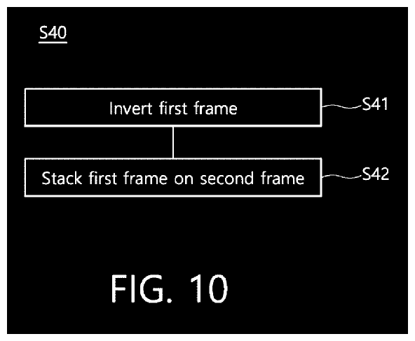

First of all, referring to FIG. 10, the operator may hold the entire first frame to which the first module in a state where the hydrogel is contracted to some extent is fixed, and then move the same adjacent to the second frame. For example, after the operator inverts the top and bottom of the first frame S41, the operator may stack the first frame in an inverted state on top of the second frame to which the second module is fixed such that the first module and the second module come into contact with each other S42. In this case, inverting the first frame by the operator is to prevent mutual connection from becoming impossible by the first module and the second module being spaced apart from each other by the side wall portion 110 of the first frame. In this way, when the entire basic frame is moved to connect the first module and the second module, the process of separating the first module from the basic frame may be omitted, and through this, it has the advantage of simplifying the process and minimizing minor damage that may occur during separation.

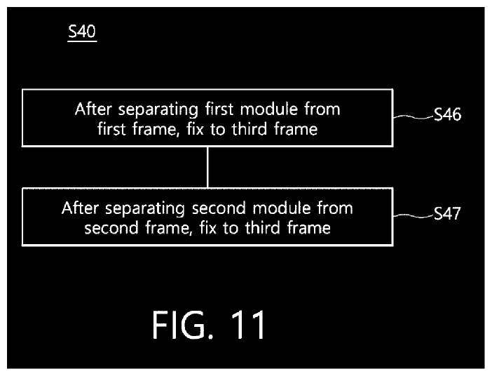

Unlike the above-described example, referring to FIG. 11, in the moving process, after separating the first module from a column portion of the first frame, as illustrated in FIG. 6, it may be mixed to a column portion 220 of the third frame 200 having a multiaxial structure S46. Thereafter, after the second module is separated from a column portion of the second frame, it may be fixed to a column portion of the third frame 200 S47. In this case, as illustrated in the drawings, the second module may be disposed in a form where a portion thereof is stacked on top of the first module. Through this, the first module and the second module may be integrated (integrally assembled) and connected to each other by contacting each other while being fixed to the column portion of the third frame 200. In this way, when the first module and the second module are moved to separate frames, a plurality of modules may be connected more effectively.

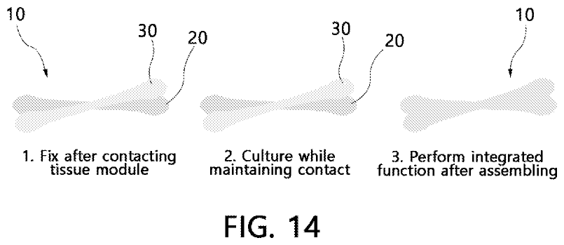

After completing the moving process, a step of inducing a portion of the hydrogel and a portion of the hydrogen constituting the first module and the second module, respectively, to be integrated S50 may be performed. That is, the first module and the second module may undergo a process of integrating (integrally assembled) by contacting each other after inducing cell alignment after cross-linking. Specifically, as illustrated in FIG. 14, tissue culture proceeds while maintaining the contact state between the first module and the second module that are in contact with each other. After 72 hours, the first module and the second module may be integrally assembled with each other to function as a single artificial muscle tissue.

In the case of forming the multiaxial artificial muscle tissue 10 by using the method (first method) of separately fabricating individual modules and integrating the same, infinite scalability may be secured in terms of production. Similar to a toy in which individual unit parts are assembled to form a block assembly having a specific shape, the manufacturer may freely connect the above-described basic modules 20, 30, 40 to produce various types of the multiaxial artificial muscle tissue 10. Particularly, in the multiaxial artificial muscle tissue 10 according to an exemplary embodiment of the present invention, each module is arranged in a form having specific directionality to reflect the characteristics of the actual muscle tissue, and by assembling the same, it is possible to adjust the contraction direction in various directions as described above.

Hereinafter, the structure 200 for forming a multiaxial artificial muscle tissue according to an exemplary embodiment of the present invention will be described. In this case, the structure 200 for forming a multiaxial artificial muscle tissue may be the third frame 200 described above. Therefore, in the following description, it will be referred to as a third frame for convenience.

Specifically, the third frame 200 may include a side wall portion 210 having a side wall to partition an accommodating space in which a predetermined amount of hydrogel is accommodated. In this case, the side wall portion 210 may be integrally formed by including a plurality of bent portions 230 that are formed by being drawn into the accommodation space as illustrated in the drawings. By limiting the shape of the hydrogel through such bent portions 230, the hydrogel may be contracted in an extended form along a desired axial direction.

In an exemplary embodiment of the present invention, the side wall portion may be formed to have a wide variety of shapes by using 3D printing technology. Through this, the multiaxial artificial muscle tissue having a more diverse multiaxial structure may be formed.

Next, a plurality of columns 221 to 224 that are supported by the side wall portion may be disposed in the accommodation space of the side wall portion. As an example, it may include a first column 221 to a fourth column 224, and in this case, a first imaginary line connecting the first column 221 and the second column 222, and a second imaginary line connecting the third column 223 and the fourth column 224 may be disposed to cross each other. In addition, the above-described bent portions 230 may be formed around a plurality of columns. Through this, the third frame may form a multi-axis A1, A2, A3 structure.

Meanwhile, the third frame 200 may be used in various ways.

First of all, when the multiaxial artificial muscle tissue is formed by the above-described first method S100, the third frame 200 may function as a fixing member to which the first to Nun modules are moved and fixed. To this end, the first column 221 and the second column 222 facing each other may be disposed to be spaced apart from each other at an interval that facilitates fixation to both end portions of the first module 20 in a state of being aligned to have directionality after cross-linking. For example, they may be disposed to be spaced apart from each other at an interval that is greater than or equal to than the interval between the column portions 120 of the basic frame 100.

In addition, when the above-described third frame 200 forms a multiaxial artificial muscle tissue by the second method, it may function as a mold to which the hydrogel is applied at once. In this case, the first column to the Nu column may function as members for applying a tensile force to the hydrogel about a plurality of axes.

Next, referring to FIG. 12, the third frame 200 may function as a fixing frame to which the entire basic frame, such as the first frame, is fixed. Specifically, as illustrated in the drawings, the frame 200 may include a side wall portion 210 including a plurality of bent portions 230 such that a plurality of basic frames 100 may be stacked and disposed in a state of crossing each other. Further, in this case, the side wall portion 210 may have a thickness that is greater than or equal to the total thickness of the stacked basic frames 100 such that a plurality of basic frames can be stacked. Through this, the side wall may guide stable entry when the basic frame 100 is inserted and disposed.

As such, the structure for forming a multiaxial artificial muscle tissue according to an exemplary embodiment of the present invention has the advantage of being able to perform various functions in a complex manner according to the method of forming a multiaxial artificial muscle tissue.

As described above, in the multiaxial artificial muscle tissue according to an exemplary embodiment of the present invention, by integrating a plurality of modules formed to contract or stretch based on a plurality of axes while maintaining their respective directionality, complex muscle tissues having various directions, such as cardiac muscle or skeletal muscle, may be effectively simulated. In particular, among the methods described above, in the case of independently forming the basic modules (first module, second module, . . . , Nu module) in charge of each part of the muscle tissue and then integrating the same (first method), it is possible to secure a wide degree of freedom in manufacturing, and thus, it is possible to form various types of muscle tissues in infinite shapes that reflect the individual characteristics of each part even within cardiac muscle or skeletal muscle.

Meanwhile, in the above description, for the convenience of explanation, although it has been described as an example that the multiaxial artificial muscle tissue simulates cardiac muscle or skeletal muscle, the application of the multiaxial artificial muscle tissue according to an exemplary embodiment of the present invention is not limited thereto, and it should be noted that it can be applied to simulate various muscles in the human body.

Although an exemplary embodiment of the present invention has been described above, the spirit of the present invention is not limited to the exemplary embodiments presented herein, and a person skilled in the art who understands the spirit of the present invention may easily suggest other exemplary embodiments by modifying, changing, deleting or adding components within the scope of the same spirit, but it can be said that this will also fall within the spirit of the present invention.

1. A multiaxial artificial muscle tissue which is a multiaxial artificial muscle tissue that is contractible or stretchable about a plurality of axes, comprising:
a first module which is formed by gelling a hydrogel including muscle cells and extends in a first axis direction so as to be contractible or stretchable in the first axis direction; and
a second module which is formed by gelling the hydrogel and extends in a second axis direction to be contractible or stretchable in the second axis direction,
wherein a portion of the hydrogel and a portion of the hydrogel which constitute the first module and the second module, respectively, are integrated with each other such that a portion of the first module and a portion of the second module are connected to each other.

2. The multiaxial artificial muscle tissue of claim 1, wherein the muscle cells include cardiomyocytes.

3. The multiaxial artificial muscle tissue of claim 1, wherein the muscle cells include skeletal muscle cells.

4. The multiaxial artificial muscle tissue of claim 1, wherein the first axis and the second axis are disposed to cross each other.

5. The multiaxial artificial muscle tissue of claim 1, wherein the first axis and the second axis are disposed to be parallel to each other.

6. The multiaxial artificial muscle tissue of claim 1, further comprising:
a third module which is formed by gelling the hydrogel and extends in a third axis direction so as to be contractible or stretchable in the third axis direction,
wherein a portion of the hydrogel and a portion of the hydrogel which constitute the first module and the third module, respectively, are integrated with each other such that a portion of the first module and a portion of the third module are connected to each other.

7. The multiaxial artificial muscle tissue of claim 1, wherein the first module and the second module are integrally formed by using a single frame in a multi-axis structure including the first axis and the second axis.

8. The multiaxial artificial muscle tissue of claim 1, wherein after the first module and the second module are primarily cross-linked at positions that are spaced apart from each other, the first module is moved to the second module and connected to each other.

9. The multiaxial artificial muscle tissue of claim 8, wherein a portion of the first module is disposed above the second module.

10. The multiaxial artificial muscle tissue of claim 8, wherein the first module and the second module are disposed to contact each other on the same plane.

11. A method for forming a multiaxial artificial muscle tissue which is a method for forming the multiaxial artificial muscle tissue according to claim 8, comprising the steps of:
forming the first module by using a first frame;
forming the second module by using a second frame which is positioned to be spaced apart from the first frame;
inducing the muscle cells included in the first module and the second module to be aligned along the first axis direction and the second axis direction, respectively;
moving the first module or the second module such that the first module and the second module come into contact with each other; and
inducing a portion of the hydrogel and a portion of the hydrogen constituting the first module and the second module, respectively, to be integrated with each other.

12. The method of claim 11, wherein the step of moving the first module to come into contact with the second module comprises the steps of:
inverting the first frame; and
stacking the first frame on the second frame such that the first module and the second module are in contact with each other.

13. The method of claim 11, wherein the step of moving the first module to come into contact with the second module comprises the steps of:
separating the first module from the first frame and fixing to a third frame; and
separating the second module from the second frame and fixing to the third frame.

14. A structure which is a structure for forming the multiaxial artificial muscle tissue according to claim 1, comprising:
a side wall portion which has a side wall to partition an accommodation space in which a predetermined amount of the hydrogel is accommodated; and
a column portion which includes first to fourth columns that are disposed to be spaced apart from each other in the accommodation space and supported by the side wall portion,
wherein the side wall portion includes a plurality of bent portions formed by being drawn into the accommodation space, and
wherein the first to fourth columns are disposed such that a first imaginary line connecting the first column and the second column and a second imaginary line connecting the third column and the fourth column cross each other.

15. The structure of claim 14, wherein the first column and the second column are disposed to be spaced apart from each other at an interval that facilitates fixation in a state where both end portions of the first module in a primarily cross-linked state are inserted, and
wherein the third column and the fourth column are disposed to be spaced apart from each other at an interval that facilitates fixation in a state where both end portions of the second module in a primarily cross-linked state are inserted.

16. A structure which is a structure for forming the multiaxial artificial muscle tissue according to claim 1, comprising:
a side wall to partition an accommodation space from the external space,
wherein the side wall includes a plurality of bent portions that are drawn into the accommodation space such that a plurality of basic frames having a rectangular shape can be stacked and disposed in a state of crossing each other in the accommodation space, and has a thickness that is greater than or equal to the stack height of the plurality of basic frames.
